(19) United States
(12) Patent Application Publication
(10) Pub. No.: US 2007/0123870 A1
(43) Pub. Date: May 31, 2007

(54) Bi-polar screw assembly

(76) Inventors: Dong Myung Jeon, Draper, UT (US); Patrick Dennis Moore, West Jordan, UT (US)

Correspondence Address: MORRISS O''BRYANT COMPAGNI, P.C., 734 EAST 200 SOUTH, SALT LAKE CITY, UT, 84102, US

(21) Appl. No.: 11/641,301

(22) Filed: Dec. 18, 2006

Related U.S. Application Data
(63) Continuation-in-part of application No. PCT/US06/09748, filed on Mar. 17, 2006.
(60) Provisional application No. 60/700,469, filed on Jul. 18, 2005.

(51) Int. Cl. A61F 2/30

(52) U.S. Cl. 606/61

(57) ABSTRACT

Assemblies, systems and components for a bi-polar bone anchor assembly. A receiver member includes a central aperture with upper and lower openings and a transverse channel. A bi-polar member and a bone anchor are loaded into the bottom of the receiver member and an internal threaded ring member fits over the outer lower threaded portion of the receiver member to retain the bi-polar member and the bone anchor therein. The bone anchor is capable of multi-axial and multi-polar positioning with respect to the receiver member. An elongated member may be placed in the channel of the receiver member in contact with the bone anchor member and a retaining member may be applied via the upper opening to press down on the elongated member thereby, locking the bone anchor member in place with the retaining member, bi-polar member, and receiver member.

### CROSS-REFERENCE TO RELATED APPLICATIONS

This application is a continuation-in-part of International Application PCT/US2006/009748 filed Mar. 17, 2006, which claims the benefit of U.S. Provisional Application No. 60/700469, filed Jul. 18, 2005. The disclosures of each of these related applications are incorporated herein by reference in their entirety.

### TECHNICAL FIELD

The present invention relates to devices and implants used in osteosynthesis and other orthopedic surgical procedures such as devices for use in spinal surgery, and, in particular, to a posterior pedicle screw, connector/rod assembly which is implantable within a patient for stabilization of the spine. Specifically, the present invention contemplates a top loading bone anchor assembly capable of achieving multiple angular, as well as multiple spherical axial orientations with respect to an elongated member extending along bone tissue.

### BACKGROUND

Several techniques and systems have been developed for correcting and stabilizing damage or malformation of bones, especially the long bones and the spine. In one type of system, an elongated member such as a bendable rod is disposed longitudinally along a length of the bone(s). In spinal applications, the rod is preferably bent to correspond to the normal curvature of the spine in the particular region being instrumented. For example, the rod can be bent to form a normal kyphotic curvature for the thoracic region of the spine, or a lordotic curvature for the lumbar region. In accordance with such a system, the rod is engaged to various vertebrae along a length of the spinal column by way of a number of fixation elements. A variety of fixation elements can be provided which are configured to engage specific portions of the vertebra and other bones. For instance, one such fixation element is a hook that is configured to engage the laminae of the vertebra. Another very prevalent fixation element is a screw that can be threaded into various parts of the vertebrae or other bones.

In one typical spinal procedure utilizing a bendable rod, the rod is situated on opposite sides of the spine or spinous processes. A plurality of bone screws are threaded into a portion of several vertebral bodies, very frequently into the pedicles of these vertebrae. The rods are affixed to this plurality of bone screws to apply corrective and stabilizing forces to the spine.

One example of a rod-type spinal fixation system includes elongated rods and a variety of hooks, screws and bolts all configured to create a segmental construct throughout the spine. In one aspect of the system, the spinal rod is connected to the various vertebral fixation elements by way of an eyebolt. In this configuration, the fixation elements are engaged to the spinal rod laterally adjacent to the rod. In another aspect of the system, a variable angle screw is engaged to the spinal rod by way of an eyebolt. The variable angle screw allows pivoting of the bone screw in a single plane parallel to the plane of the spinal rod. Details of this variable angle screw can be found in U.S. Pat. No. 5,261,909 to Sutterlin et al. One goal achieved by the system is that the surgeon can apply vertebral fixation elements, such as a spinal hook or a bone screw, to the spine in appropriate anatomic positions. The system also allows the surgeon to easily engage a bent spinal rod to each of the fixation elements for final tightening.

Another rod-type fixation system provides a variety of fixation elements for engagement between an elongated rod and the spine. In one aspect of the system, the fixation elements themselves include a body that defines a slot within which the spinal rod is received. The slot includes a threaded bore into which a threaded plug is engaged to clamp the rod within the body of the fixation element. The system includes hooks and bone screws with this “open-back” configuration. Details of this technology can be found in U.S. Pat. No. 5,005,562.

On the other hand, these fixation elements of the system are capable only of pivoting about the spinal rod to achieve variable angular positions relative to the rod. While this limited range of relative angular positioning is acceptable for many spinal pathologies, many other cases require more creative orientation of a bone screw, for instance, relative to a spinal rod. Certain aspects of this problem are addressed by the variable angle screw of the system, as discussed in the '909 Patent. However, there is a need for a bone screw that is capable of angular orientation in multiple planes relative to the spinal rod as well as multiple spherical head orientations. Preferably, the bone screw axis is capable of various three dimensional orientations with respect to the spinal rod, as well as three dimensional spherical axis orientation to the receiving (head) element of the device's axial orientation of the bone engaging screw member. Screws of this type of angular orientation in multiple planes relative to the spinal rod have been referred to as poly-axial or multi-axial bone screws. One should note, as of yet, no known screw systems have employed both angular orientation in multiple planes relative to the spinal rod and three dimensional spherical axis orientation to the receiving (head) element of the device's axial orientation of the bone engaging screw member. The use of both angular orientation in multiple planes relative to the spinal rod and three dimensional spherical axis orientation to the receiving (head) element of the device's axial orientation of the bone engaging screw member technology allows for virtually unlimited axial angulations of the bone engaging screw member as well as an ultra-low profile of the said device utilizing a minimum of components without sacrificing the security of the interfaces of the invention components.

Others have approached the solution to this problem with various poly-axial screw designs. For example, in U.S. Pat. No. 5,466,237 to Byrd et al., a bone screw is described which includes a spherical projection on the top of the bone screw. An externally threaded receiver member supports the bone screw and a spinal rod on top of the spherical projection. An outer nut is tightened onto the receiver member to press the spinal rod against the spherical projection to accommodate various angular orientations of the bone screw relative to the rod. While this particular approach utilizes a minimum of components, the security of the fixation of the bone screw to the rod is lacking. In other words, the engagement or fixation between the small spherical projection on the bone screw and the spinal rod is readily disrupted when the instrumentation is subjected to the high loads of the spine, particularly in the lumbar region.

In another approach shown in U.S. Pat. No. 4,946,458 to Harms et al., a spherical headed bone screw is supported within separate halves of a receiver member. The bottom of the halves are held together by a retaining ring. The top of the receiver halves are compressed about the bone screw by nuts threaded onto a threaded spinal rod. In another approach taken by Harms et al. in U.S. Pat. No. 5,207,678, a receiver member is flexibly connected about a partially spherical head of a bone screw. Conical nuts on opposite sides of the receiver member are threaded onto a threaded rod passing through the receiver. As the conical nuts are threaded toward each other, the receiver member flexibly compresses around the head of the bone screw to clamp the bone screw in its variable angular position. One detriment of the systems in the two Harms et al. patents is that the spinal rod must be threaded in order to accept the compression nuts. It is known that threading rods can tend to weaken the rods in the face of severe spinal loads. Moreover, the design of the bone screws in the '458 and '678 Patents require a multiplicity of parts and are fairly complicated to achieve complete fixation of the bone screw.

A further approach illustrated in U.S. Pat. No. 5,797,911 to Sherman et al. is to provide a U-shaped holder through the top of which a bone fastener topped with a crown member is loaded. The holder accommodates a rod in a channel above the crown member and a compression member above the rod. The compression member presses on the rod and crown member to lock the fastener against the holder in any one of a number of angles in three dimensions with respect to the rod. This approach has proven to be quite effective in addressing the above-identified problems. However, it does not permit bottom-loading of the fastener. Additionally, the holder is somewhat bulky in order to accommodate the other structural components.

Yet a further approach is shown in U.S. Pat. No. 5,733,285 to Errico et al., in which a holder is provided with a tapered and colletted portion at the bottom into which a bone fastener head is inserted. A sleeve is provided that slides down around the colletted portion to crush lock the colletted portion around the head of the bone fastener. This apparatus is believed to be relatively bulky and difficult to manipulate given the external sliding locking mechanism. It is further dependent on the fit of the external sleeve and the relative strength of the collet and its bending and crushing portions for secure locking of the bone fastener head.

There is therefore a need remaining in the industry for an ultra-low profile, multi-axial/bi-polar bone anchor that can be readily and securely engaged to an elongated member of any configuration—i.e., smooth, roughened, knurled or even threaded--which achieves greatly improved angulations of the bone anchor, improved strength, and reduced size, including profile and bulk, of the components used to engage the bone anchor to the elongated member in any of a variety of angular orientations.

### SUMMARY

In one embodiment of the invention, a bone fixation assembly is provided that includes a receiver member defining an upper opening portion and a lower opening portion each having respective minimum widths, a channel configured to receive an elongated member (rod) and communicating with said upper opening portion and said lower opening portion, and a bi-polar member having an internal portion configured to engage a bone anchor head and an external portion configured to engage the internal geometry of the receiver member, said internal width of said bi-polar member being larger than said width of the head of the bone-anchor member and said external width of said bi-polar member larger than said minimum width of said lower opening portion of said internal threaded ring member, said head of the bone-anchor member being movably disposed in said lower opening portion adjacent to said internal surface of said bi-polar member; and a bone-engaging anchor having a lower portion configured to engage a bone and a head having a width, said width of said head being smaller than said minimum width of said lower opening portion, said head being movably disposed in said lower opening portion adjacent to said lower surface of said bi-polar member; and an ring member that fits around the bone anchor and over the outer lower portion of the receiver member to retain the bi-polar member and the bone anchor member.

Once the bone anchor member and bi-polar member are retained in the lower opening of the receiving member, the bi-polar and the bone anchor member is capable of multi-axial positioning as well as multi-polar positioning with respect to the receiver member. A compression retaining member defining an aperture smaller than said width of said head, may be at least partially housed in said internally threaded portion of said receiver member and positioned over said elongated member and then tightened down against an inserted rod. Forces transmitted during tightening are imparted on the bone anchor member, bi-polar member, and the lower surface of the receiving member and the ring member to anchor all the components in any angular and/or axial configuration within design parameters.

Additional embodiments, examples, advantages, and objects of the present invention will be apparent to those of ordinary skill in the art from the following specification.

### DESCRIPTION OF THE DRAWINGS

It will be appreciated by those of ordinary skill in the art that the elements depicted in the various drawings are not to scale, but are for illustrative purposes only. The nature of the present invention, as well as other embodiments of the present invention may be more clearly understood by reference to the following detailed description of the invention, to the appended claims, and to the several drawings attached hereto.

FIG. 1 is a side elevational view of one embodiment of the multi-axial bone screw anchor assembly of the present invention.

FIG. 2 is an exploded view of the embodiment of the invention depicted in FIG. 1.

FIG. 3A is a side elevational view of an embodiment of the receiver member of the embodiment of the invention illustrated in FIG. 2.

FIG. 3B is a front elevational view of the embodiment of the receiver member illustrated in FIG. 3A.

FIG. 3C is a sectional view, taken along the lines 3C-3C in FIG. 3B, and viewed in the direction of the arrows, of the embodiment of the receiver member illustrated in FIG. 3A.

FIG. 3D is a sectional view, taken along the lines 3D-3D of FIG. 3B and viewed in the direction of the arrows, of the embodiment of the receiver member illustrated in FIG. 3A.

FIG. 4A is a side elevational view of an embodiment of a bone anchor used in the embodiment of the invention illustrated in FIG. 2.

FIG. 4B is a sectional view, taken along the lines 4B-B of FIG. 4A and viewed in the direction of the arrows, of the embodiment of the bone anchor illustrated in FIG. 4A.

FIG. 4C is a magnified view of one embodiment of the head of the embodiment of the bone anchor illustrated in FIG. 4A.

FIG. 5A is a top view of one embodiment of a bi-polar member used in the embodiment of the present invention illustrated in FIG. 2.

FIG. 5B is a sectional view, taken along the lines 5B-5B in FIG. 5A and viewed in the direction of the arrows, of the embodiment of the bi-polar member illustrated in FIG. 5A.

FIG. 5C is a sectional view substantially similar to FIG. 5B of another embodiment of a bi-polar member used in the embodiment of the invention illustrated in FIG. 2.

FIG. 6A is a top view of one embodiment of an internal threaded ring member that fits around the bone anchor and over the outer lower threaded portion in the receiver member to retain the bi-polar member and the bone anchor member used in the embodiment of the invention illustrated in FIG. 2.

FIG. 6B is a sectional view, taken along the lines of 6B-B in FIG. 6A and viewed in the direction of the arrows, of the embodiment of the internal threaded ring member illustrated in FIG. 6A.

FIG. 7A is a top view of a retaining member for use with some embodiments of the present invention.

FIG. 7B is a side elevational view of the retaining member of FIG. 7A.

FIG. 8 is an enlarged sectional view of one illustrative embodiment of an assembled system in accordance with the present invention, including the components illustrated in FIGS. 1, 2, 7A, and 7B.

### DETAILED DESCRIPTION

For the purposes of promoting an understanding of the principles of the invention, reference will now be made to the embodiment illustrated in the drawings and specific language will be used to describe the same. It will nevertheless be understood that no limitation of the scope of the invention is thereby intended, such alterations and further modifications in the illustrated device, and such further applications of the principles of the invention as illustrated therein, being contemplated as would normally occur to one skilled in the art to which the invention relates.

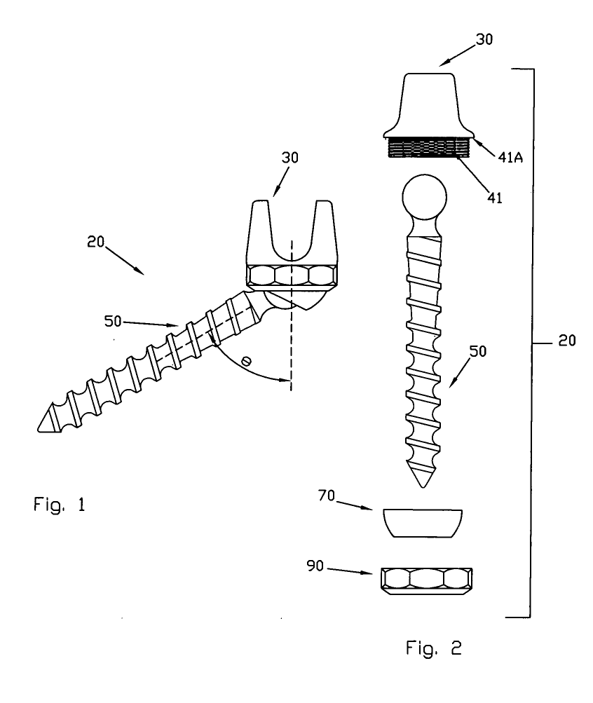

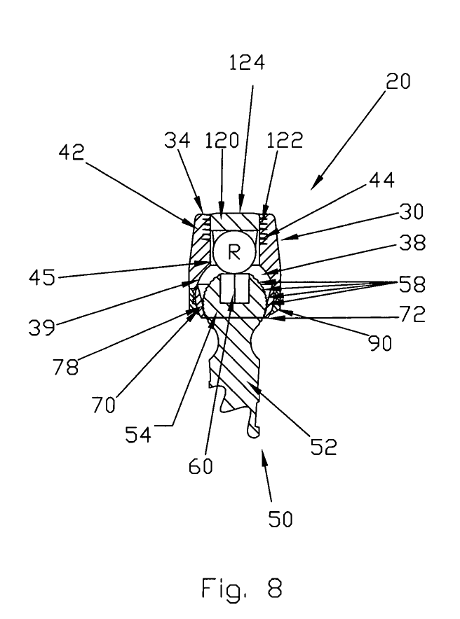

Referring generally to FIGS. 1 and 2, there is shown one embodiment of a multi-axial/bi-polar bone anchor assembly 20 in accordance with the principles of the present invention. In the illustrated embodiment, assembly 20 includes a receiver member 30, a bone anchor 50, a bi-polar member 70, and an internal threaded ring member 90. The assembly 20 of the present invention is designed for use with an elongated member R (depicted in FIG. 8) such as a spinal rod, bar or other orthopedic construct, as further described below.

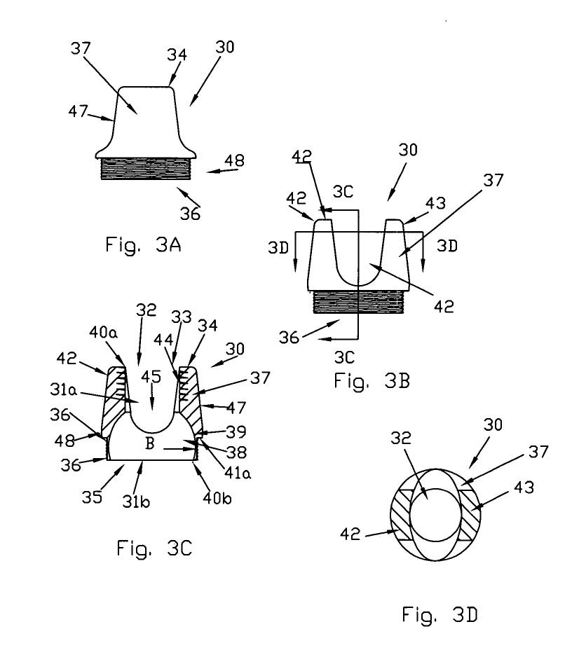

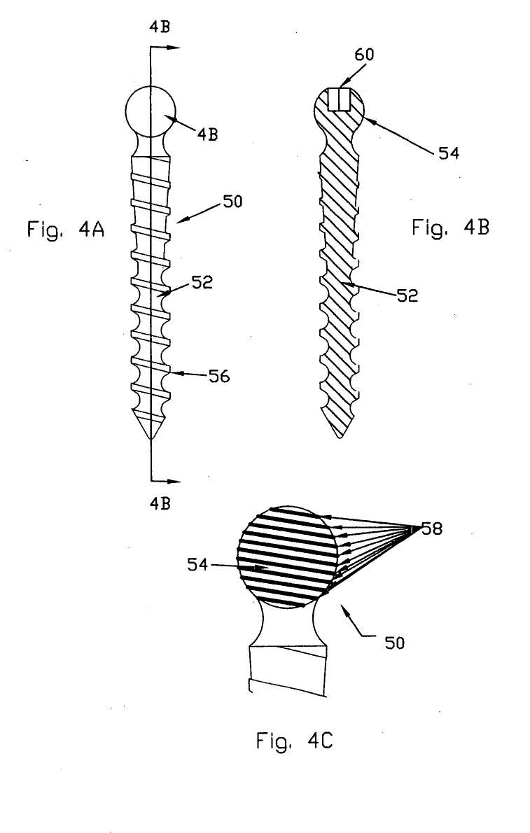

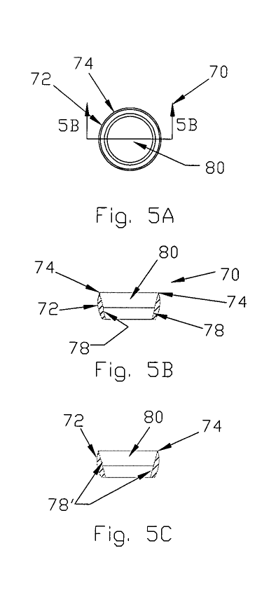

Referring now generally to FIGS. 3A-3D, additional details of one illustrative embodiment of a receiver member 30 in accordance with the present invention are shown. Receiver member 30 is formed as a generally circular member having at least one sidewall 33 surrounding a central aperture 32. Sidewall 37 defines an upper portion 47 including top end 34 and a lower portion 48 including bottom end 36. Central aperture 32 extends through receiver member 30 from an upper aperture 33 in top end 34 to a lower aperture 35 in bottom end 36. Lower portion 31b of central aperture 32, in one specific embodiment, includes a chamber/void 38 defined by a chamber wall 39 which is configured to form a spherical chamber. Alternatively, central aperture in upper and lower portions 31 a and 31 b can have a variety of configurations, such as each having one or more sections of differing diameter.

Central aperture 32 includes a top portion 31a which may be partially surrounded by a chamfered or rounded edge 40a at top end 34 of receiver member 30. Similarly, bottom portion 31b of central aperture 32 may be surrounded by a chamfered or rounded edge 40b at the bottom end 36 of receiver member 30. Proximate to bottom end 36, receiver member 30 may define external threads 41 and an associated ledge 41a (FIG. 2C). In the illustrated embodiment, threads 41 extends around the entire perimeter of lower surface 31b, although it will be seen that thread 41 could extend only partially around the perimeter of lower surface 31b.

Sidewall 33 of receiver member 30 may define one or more pairs of upright branches 42, 43 in upper portion 31a through which central aperture 32 extends. Branches 42, 43 further define one or more channels, such as U-shaped channel 45, which extend transversely to central aperture 32, and that may accommodate an elongated member R (FIG. 8) therein. In one specific embodiment, internal threads 44 may be formed in branches 42 and 43 in the sidewall of central aperture 32. These internal threads 44 may be a modified acme buttress thread or other suitable thread. In other embodiments, the branches 42, 43 may feature an external thread. The top portion 47 of receiver member 30 (which includes branches 42, 43) may be narrower than bottom portion 48 of receiver member 30 to thereby reduce the bulk and profile of receiver member 30.

Referring now generally to FIGS. 4A-4C, one illustrative embodiment of a bone anchor 50 which may be used in the present invention is depicted. The illustrated bone anchor 50 is a bone screw. Bone anchor 50 includes an anchorage portion 52 and a head portion 54. Anchorage portion 52 is formed as a shaft including at least one thread 56, which may be a cancellous self-tapping thread. Head portion 54 is disposed at a proximal end of the anchorage portion 52 and forms part of a sphere in the illustrated embodiment, though alternative curvate and other configurations may be employed. In some embodiments, head 54 may include a series of ridges 58 for improving purchase with the inside of bi-polar member 70 (described below). Head 54 may have alternative friction-increasing surface configuration(s) such as roughening or knurling. Further, head 54 includes a tool-engaging print 60, with which a tool (not shown) may be engaged to drive anchorage portion 52 into a bone. Tool-engaging print 60 is an interior print in the illustrated embodiment, although an exterior print could be used, and it may have any of a number of configurations, such as hexagonal, hexalobate, X-shaped, or other known torque-transferring configurations.

Other embodiments of bone anchor 50 are contemplated as being within the scope of the present invention. For example, bone anchor 50 could be a bone-engaging hook rather than a screw. In such embodiments, anchorage portion 52 may be configured with a hook rather than an elongated section with thread 56.

Head 54 of bone anchor 50 is shaped and sized to fit within at least interior portion 78 of bi-polar member 70 (depicted in FIGS. 5A-5C) and chamber 38 of receiver member 30 (FIG. 3C). Specifically, head 54 has a width that is smaller than the width of bi-polar member 70 and chamber 38. As more fully described below, bone anchor 50 is inserted into receiver member 30, with anchorage portion 50 entering through opening 80 and interfacing with surface 78 of bi-polar member 70 (FIG. 5A).

Referring now to FIGS. 5A-5C, there is shown one illustrative embodiment of bi-polar member 70 in accordance with the principles of the present invention. In the depicted embodiment, bi-polar member 70 is formed as a circular disc, having an exterior surface 72 with a beveled edge 74 and an interior surface 78. Interior surface 78 is configured to accommodate head 54 of bone anchor 50. Accordingly, the illustrated embodiment of interior surface 78 has the shape of part of a sphere. It will be appreciated that in other embodiments, the shape may differ, in order to accommodate other head 54 shapes. For example, see the conical interior surface 78′ of FIG. 5C. Interior surface 78 can be provided with a friction or purchase-enhancing surface configuration (e.g. roughening or knurling) for cooperation with head 54 of bone anchor 50.

Bi-polar member 70 also includes a hole 80 faced by interior surface 78. Hole 80 is provided so that bone anchor 50 may be partially passed therethrough, allowing the bone engaging threads 56 of bone anchor 50 to be available through bi-polar member 70, while head 54 is retained therein. The dimension of hole 80 of the bi-polar member 70 is preferably slightly larger than the outer dimension of bone anchor head 54 so that the bone anchor head 54 is slidably and rotatably movable within hole 80 and bipolar member 70.

Bi-polar member 70 is sized and shaped to fit within at least lower portion 31b of central aperture 32 and chamber 38 of receiver member 30. The outer dimension of bi-polar member 70 is preferably slightly smaller than the inner dimension of chamber 38 and lower portion 31b of central aperture 32 so that bi-polar member 70 is slidably and rotatably movable within chamber 38 and central aperture 32. Further, in the illustrated embodiment, the outer dimension of bi-polar member 70 is larger than the inner dimension of upper opening portion 31a, so that bi-polar member 70 cannot move into upper opening portion 31a.

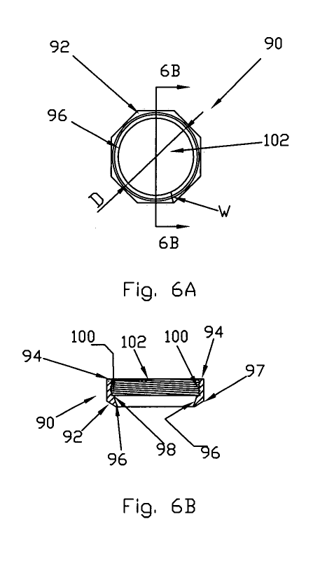

Referring now to FIGS. 6A-6B, there is depicted one illustrative embodiment of an internal threaded ring member 90 in accordance with the principles of the present invention. In the illustrated embodiment, internal threaded ring member 90 may be formed as a generally ring-shaped component including a bottom surface 92 and a top surface 94. In the illustrated embodiment on one internal threaded ring member 90, an internal surface 91 surrounds aperture 102 and includes a number of structures. The lower portion 96 of internal surface 91 forms a portion of a sphere of radius substantially identical to the radius of head 54 of bone anchor 50, above which a medial portion 98 is generally cylindrical and an upper portion 100 is conical and angled outward to allow a greater range of angular positioning of an inserted bone anchor 50. In alternative embodiments, the internal surface 91 may have single or multiple surface configurations, which may be cylindrical, conical, spherical or of other appropriate configuration. The diameter of aperture 102 is smaller than the diameter of head 54 of bone anchor 50 and the diameter of bi-polar member 70.

As depicted, the external surface 97 of the internal threaded ring member 90 may have a polygonal shape, such as rectangular or octagonal shape for interaction with a securing tool, such as a wrench.

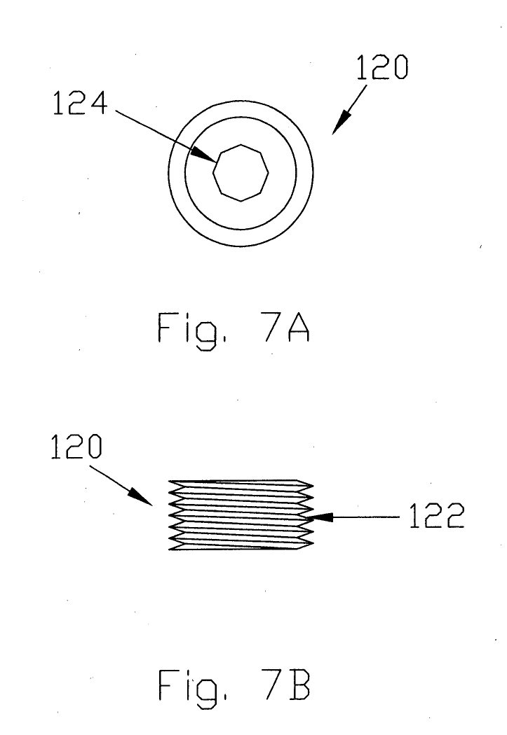

FIGS. 7A and 7B depict one illustrative embodiment of a retaining member or compression member 120 in accordance with the principles of the present invention. As depicted, retaining member 120 may be a set screw or threaded plug having external threads 122 and a print 124 for interaction with a tool (not shown) for applying torque. In assembly, retaining member 120 may be threaded into threads 44 of receiver member 30 (FIG. 3C) and down onto an inserted elongated member R (FIG. 8). In one alternative embodiment, where receiver member 30 is externally threaded, compression member 120 could be an internally-threaded nut.

Generally referring to FIGS. 1, 2 and 8, assembly 20 may be assembled together by inserting a bone anchor 50 through a bi-polar member 70 and an internal threaded ring member 90, then inserting the head 54 of the bone anchor and bi-polar member 70 into receiver member 30 through bottom end 36. This may occur as a series of individual steps or may be substantially in one step as shown in (FIG. 2). Internal threaded ring member 90 may then be rotated to secure the components to one another.

Bi-polar member 70 remains slidably and rotatably positioned in lower portion 31b of central aperture 32 and/or chamber 38 of receiving member 30, and bone anchor 50 remains multi-axially moveable with respect to bi-polar member 70 and receiving member 30. Internal threaded ring member 90 is threaded upward into lower portion 48 of receiver member 30.

When internal threaded ring 90 is installed, bone anchor 50 and bi-polar member 70 are retained within central aperture 32 of receiver member 30. The head 54 of bone anchor 50 is supported by bi-polar member 70, and bi-polar member 70 is supported by the internal surface 96 of internal threaded ring member 90. Thus bone anchor 50 and bi-polar member 70 will not pass through internal threaded ring 90 and out of receiver member 30 once the internal threaded ring 90 is installed.

Assembly 20 may be assembled to this point prior to use in a surgical procedure. During installation, bone anchor 50 of assembly 20 is attached to an appropriately prepared bone (not shown). With the depicted embodiment, this may be by threading the bone anchor 50 into a predrilled hole in the bone. Threaded anchoring portion 52 is inserted into the hole, and an appropriate screwing tool may be used with tool-engaging print 60 of bone anchor 50, and bone anchor 50 is threaded into the bone. When bone anchor 50 has been threaded into the bone to the desired depth, receiver member 30 is positioned so that central aperture 32 forms a desired angle with bone anchor 50, as depicted in FIG. 1. In alternative embodiments, for example where bone anchor 50 is a bone hook, drilling a hole in bone and threading the anchor therein may not be necessary.

In the illustrated embodiment, the angle theta (FIG. 1) between bone anchor 50 and central aperture 32 can be any value up to about 57 degrees in any direction (up to about 112 degrees total angulation). It will be seen that the angle of bone anchor 50 relative to opening 32 can be changed in two ways. First, the angle of bone anchor 50 with respect to the bi-polar component 70 may be adjusted. Second, the angle of the bipolar component 70 with respect to the receiver member 30 can be adjusted.

As described above, receiver member 30 may be angled as the surgeon desires with respect to bone anchor 50. An elongated member R such as a spinal rod, connector, or other orthopedic surgical implant may be coupled with assembly 20. Elongated member R may be placed in channel 45 of receiver member 30 and contact interior surface 78 of bi-polar member 70. A retaining member or compression member 120, such as a set screw or threaded plug, may be threaded into threads 44 of receiver member 30 and down onto elongated member R. As compression member 120 is tightened, elongated member R is forced downward against bone anchor 50 and bi-polar member 70, pushing bi-polar member 70 down onto head 54 of bone anchor 50. Head 54 is thereby clamped between internal threaded ring member 90 and bi-polar member 70. In the embodiment of the invention in which head 54 includes ridges 58, ridges 58 are pressed into internal surface 78 of bi-polar member 70. In this way, bone anchor 50 and bi-polar member 70 are locked into the desired angular position with respect to elongated member R and the remainder of assembly 20.

It will be appreciated that where appropriate and desired, the assembly 20 can be assembled during the surgical procedure.

Components of assembly 20 may be constructed of any surgically acceptable material of sufficient strength to be used to retain elongated member R. For example, stainless steel, titanium, and their alloys can be used. It will be appreciated that any sturdy biocompatible material may be used to accomplish the osteosynthesis and other orthopedic surgical goals of the present invention.

While the present invention has been shown and described in terms of preferred embodiments thereof, it will be understood that this invention is not limited to any particular embodiment and that changes and modifications may be made without departing from the true spirit and scope of the invention as defined and desired to be protected.

What is claimed is:

1. An assembly for securing an elongated member for surgical stabilization of a bone, the assembly comprising:
a bone anchor comprising a bone engaging portion extending from a curvate head;
a bi-polar member, comprising a circular disc having a beveled exterior and an aperture extending from a top opening to a bottom opening, the top opening having a greater diameter than the bottom opening such that the bone anchor may be inserted partially therethrough with the bone engaging portion passing out the bottom opening and the head of a bone anchor retained in the aperture adjacent at least open sidewall thereof;
a receiver member comprising at least one sidewall defining a central channel passing from a first opening at a top end to a second opening at a bottom end, the central channel having an enlarged lower portion adjacent the bottom end sized to receive the bi-polar member with the head of an inserted bone anchor therein, and at least one transverse channel formed in an upper portion of the receiver member generally perpendicular to the central channel, the at least one transverse channel formed as two opposite slots extending from the top end of the receiver member; and
a lower retaining member comprising a generally ring-shaped member having a central aperture with an upper opening at a top surface and a smaller lower opening at a lower surface and an attachment structure for attachment at a lower portion of the receiver member.

2. The assembly of claim 1, wherein the bone engaging portion of the bone anchor comprises a threaded shaft.

3. The assembly of claim 2, wherein the threaded shaft comprises a cancellous self-tapping thread.

4. The assembly of claim 1, wherein the curvate head of the bone anchor has a generally spherical shape.

5. The assembly of claim 1, wherein the curvate head of the bone anchor features a series of ridges or grooves.

6. The assembly of claim 1, wherein the curvate head of the bone anchor includes a tool-engaging print.

7. The assembly of claim 1, wherein the beveled exterior of the bi-polar member has a generally spherical shape.

8. The assembly of claim 1, wherein the aperture of the bi-polar member has a generally spherical shape.

9. The assembly of claim 1, wherein the at least one sidewall of the bi-polar member has a roughened or knurled surface.

10. The assembly of claim 1, wherein the receiver member further comprises a threading in the top portion of the central channel for interaction with a cylindrical threaded plug inserted therein.

11. The assembly of claim 10, wherein threadably inserting a threaded plug into the top portion of the central channel will compress an elongated retaining member inserted into the transverse channel against the head of an inserted bone anchor, compressing the head of the inserted bone anchor against the bi-polar member and the bi-polar member against the lower retaining member to retain the bone anchor in a desired angular position.

12. The assembly of claim 1, wherein the receiver member further comprises an external threading disposed adjacent a bottom portion thereof for attachment to the lower retaining member.

13. The assembly of claim 12, wherein the attachment structure on the lower retaining member comprises an internal threading on a sidewall of the central aperture for interaction with the external threading on the bottom portion of the receiver member.

14. The assembly of claim 1, wherein the lower retaining member has a polygonal shape.

15. The assembly of claim 1, wherein the lower opening and central aperture of the lower retaining member allow angular positioning of the bi-polar member with respect thereto until compression of the head of the bone anchor by an elongated member secured in the transverse channel.

16. The assembly of claim 15, wherein the central aperture of the lower retaining member has a concave surface for engaging an external surface of the bi-polar member.

17. A bone anchor system for securing a spinal rod, the system comprising:
a receiver having a body with an upper portion and a lower portion, a central channel passing from a first opening at a top end to a second opening at a bottom end, the central channel having a first width in the upper portion and a second width larger than the first width in the lower portion adjacent the bottom end, and a transverse channel formed in an upper portion by opposing slots extending from the top end of the receiver, the transverse channel being generally perpendicular to the central channel and a width sufficient to receive a spinal rod inserted therein;
a bi-polar disc comprising a circular body with a beveled exterior and an aperture extending from a top opening to a bottom opening, the top opening having a greater diameter than the bottom opening, the circular body having a top diameter larger than the first width and smaller than the second width of the central channel of the receiver;
a bone anchor comprising a bone engaging portion extending from a curvate head, the curvate head having a maximum width smaller than the top opening of the bi-polar disc and larger than the bottom opening of the bi-polar disc; and
a lower retainer comprising a generally ring-shaped body with a central aperture and an attachment structure for attachment to the lower portion of the receiver.

18. The system of claim 17, further comprising a generally cylindrical plug with external threading for attachment to an internal threading in the upper portion of the receiver to thereby retain an inserted spinal rod in the transverse channel.

19. The system of claim 17, wherein the second width of the central channel of the receiver body is a maximum width of a generally spherical chamber in the lower portion of the receiver.

20. The system of claim 19, wherein the beveled exterior of the bi-polar disc has a generally spherical curve corresponding to the generally spherical chamber of the central channel of the receiver.

21. The system of claim 17, wherein the aperture of the bi-polar disc is formed as a curved sidewall.

22. The system of claim 17, wherein the bone engaging portion of the bone anchor comprises a threaded shaft.

23. The system of claim 17, wherein the curvate head of the bone anchor has a generally spherical shape.

24. The system of claim 17, wherein the curvate head of the bone anchor has a series of ridges or grooves.

25. The system of claim 17, wherein the interior surface and exterior surface of the bi-polar disc are roughened or knurled.

26. The system of claim 17, wherein the receiver further comprises an external threading on the side of bottom portion for attachment to the lower retainer.

27. The system of claim 27, wherein the lower retainer has a polygonal shape.

28. The system of claim 17, wherein the central aperture of the lower retainer has a concave surface.
